(19) United States
(12) Patent Application Publication
(10) Pub. No.: US 2023/0310474 A1
(43) Pub. Date: Oct. 5, 2023

(54) CORONAVIRUS TREATMENT COMPOSITION AND METHOD

(71) Applicants: Cleveland State University, Cleveland, OH (US); University of Tennessee Research Foundation, Knoxville, TN (US)

(72) Inventors: Xue-Long Sun, Solon, OH (US); Guoyun Chen, Memphis, TN (US)

(73) Assignees: Cleveland State University, Cleveland, OH (US); University of Tennessee Research Foundation, Knoxville, TN (US)

(21) Appl. No.: 18/109,021

(22) Filed: Feb. 13, 2023

Related U.S. Application Data
(60) Provisional application No. 63/309,117, filed on Feb. 11, 2022.

(51) Int. Cl. A61K 31/7028; A61P 31/14

(52) U.S. Cl. CPC A61K 31/7028; A61P 31/14

(57) ABSTRACT

An antiviral composition contains a sialidase inhibitor. Methods for treating a viral infection include administering the composition to a patient. The sialidase inhibitor may be of general formula (l)

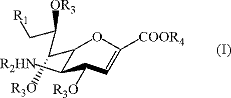

wherein R₁ is selected from the group consisting of OH, O-acetyl, N₃, OCOCH₂X₁ and NHCOCHX₁; X₁ is selected from the group consisting of H, a C₁-C₅ alkyl group, acetyl, propionyl, butyryl, valoryl, hexanoyl, pentanoyl, and 2-methylpropanyl; R₂ is H or COCH₂X₂; X₂ is selected from the group consisting of H, a C₁-C₅ alkyl group, acetyl, propionyl, butyryl, valoryl, hexanoyl, pentanoyl, and 2-methylpropanyl; R₃ is selected from the group consisting of H, OH, COCH₂X₃ and OCOCH₂X₃; X₃ is selected from the group consisting of H, a C₁-C₅ alkyl group, acetyl, propionyl, butyryl, valoryl, hexanoyl, pentanoyl, and 2-methylpropanoal; R₄ is selected from the group consisting of H, OH, a C₁-C₆ alkyl group, and CH₂X₄; and X₄ is H or a C₁-C₆ alkyl group.

### CROSS REFERENCE TO RELATED APPLICATIONS

This application claims the benefit of U.S. Provisional Application No. 63/309,117 filed Feb. 11, 2022 and titled “ANTIVIRAL COMPOSITIONS AND TREATMENT METHODS”, which is incorporated by reference in its entirety.

### STATEMENT REGARDING FEDERALLY SPONSORED RESEARCH OR

### DEVELOPMENT

This invention was made with government support under Grant No. AI137255 awarded by the National Institutes of Health (NIH). The government has certain rights in the invention.

### BACKGROUND

Coronavirus disease 2019 (COVID-19) is caused by severe acute respiratory syndrome coronavirus 2 (SARS-CoV-2). To date, there are no generally effective therapies for COVID-19 or antivirals against SARS-CoV-2. Further compounding this issue, current vaccines appear less efficacious against new variants of the virus. There is a need for new antiviral compositions and treatment methods.

### BRIEF DESCRIPTION

Disclosed, in some embodiments, is a sialidase inhibitor of general formula (I):

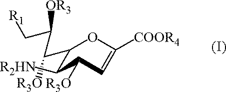

wherein R₁ is selected from the group consisting of OH, O-acetyl, N₃, OCOCH₂X₁ and NHCOCHX₁; X₁ is selected from the group consisting of H, a C₁-C₅ alkyl group, acetyl, propionyl, butyryl, valoryl, hexanoyl, pentanoyl, and 2-methylpropanyl; R₂ is H, acetyl, or COCH₂X₂; X₂ is selected from the group consisting of H, a C₁-C₅ alkyl group, acetyl, propionyl, butyryl, valoryl, hexanoyl, pentanoyl, and 2-methylpropanyl; R₃ is selected from the group consisting of H, OH, acetyl, COCH₂X₃ and OCOCH₂X₃; X₃ is selected from the group consisting of H, a C₁-C₅ alkyl group, acetyl, propionyl, butyryl, valoryl, hexanoyl, pentanoyl, and 2-methylpropanoal; R₄ is selected from the group consisting of H, OH, a C₁-C₆ alkyl group, and CH2X₄; and X₄ is H or a C₁-C₆ alkyl group.

The sialidase inhibitor may be of the formula:

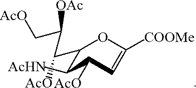

Disclosed, in other embodiments, is a treatment composition containing a carrier and a sialidase inhibitor. The sialidase inhibitor in the composition may be of general formula (I) or the specific formula above.

In some embodiments, the sialidase inhibitor is a membrane-permeable sialidase inhibitor.

The sialidase inhibitor may be a cytosolic sialidase inhibitor.

In some embodiments, the sialidase inhibitor is hydrophobic and lipophilic.

Disclosed, in further embodiments, is a method for treating a viral infection which includes administering an effective dose of an antiviral composition containing a sialidase inhibitor to a patient.

The composition may be administered orally, via an injection, intraperitoneally, and/or intravenously.

In some embodiments, the viral infection is a coronavirus infection (e.g., a severe acute respiratory syndrome coronavirus 2 (SARS-CoV-2) infection).

The sialidase inhibitor may be selected from Neu5Gc2en, Neu5Ac2en9N3, Neu5Ac2en-OMe, Neu5Ac2en9N3-OAc, Neu5Ac2en-OAcOMe, and Neu5Ac2en9N3-OAcOMe. It is also contemplated that the composition may contain more than one sialidase inhibitor.

In particular embodiments, the sialidase inhibitor is Neu5Ac2en-OAcOMe.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIGS. 1A-L ilustrate that sialylation on coronavirus nucleocapsid (N) protein is critical for its RNA binding activity and viral replication. FIGS. 1A-C show immunoprecipitated concentration of nucleocapsid protein using the sera of COVID19 patients (FIG. 1A), cell lysates from HCoV-OC43-infected THP-1 cells (FIG. 1B), or HEK293T cell lysates overexpressing SARS-CoV-2 nucleocapsid (FIG. 1C), followed by immunoblot analysis of sialylation using biotin-MAA (α2,3-linkage), biotin-SNA (α2,6-linkage) lectins, biotin-anti-SARS-CoV-2-N (a), anti-HCoV-OC43-N or anti-SARS-CoV-2-N antibodies (c). FIGS. 1D and 1E show gel mobility shift assay of the 32-mer ssRNA (FIG. 1D) or 32-mer ssDNA (FIG. 1E). The probe was incubated with no cell lysates (lane 1), or lysates with the treatment indicated (lanes 2-5). FIG. 1F shows the mRNA levels of Neu1, Neu2, Neu3 and Neu4 normalized by GAPDH in THP-1 cells with or without HCoV-OC43 infection for 72 hours. FIG. 1G shows immunoblot analysis of Neu1 in naïve and HCoV-OC43 infected THP-1 cells. β-actin was used as the internal control. FIG. 1H shows N protein associated with endogenous Neu1 in HCoV-OC43-infected THP-1 cells (48 hours post-infection). FIG. 1l shows immunoblot analysis of Neu1 and viral nucleocapsid in Neu1 overexpressing THP-1 cells. FIG. 1J shows the levels of intracellular viral RNA (upper) and extracellular viral titers (lower) in control and Neu1 overexpressing THP-1 cells. FIG. 1K shows immunoblot analysis of Neu1 and viral nucleocapsid in scrambled and Neu1 knockdown THP-1 cells. FIG. 1L shows the levels of intracellular viral RNA (upper) and extracellular viral titers (lower) in scrambled and Neu1 knockdown THP-1 cells. Data are representative of three (FIGS. 1A-H) or two (FIG. 11-L) independent experiments. Data are shown as mean ± SD. *p < 0.05; **p < 0.01; ***p < 0.001. Analysis was performed using two-way ANOVA.

FIGS. 2A-C illustrate the results of screening for sialidase inhibitors suppressing coronavirus propagation. FIGS. 2A and 2B show the intracellular viral RNA levels (upper) and viral titers (lower) in the supernatant of HCoV-OC43 (MOI = 2) infected THP-1 cells treated with sialidase inhibitors Neu5Gc2en, zanamivir, Neu5Ac2en9N3, Neu5Ac2en-OMe, Neu5Ac2en-OAcOMe, Neu5Ac2en9N3-OAcOMe (FIG. 2A) or oseltamivir (FIG. 2B). FIG. 2C shows the Vero 76 cells were treated with the above 7 sialidase inhibitors and infected with SARS-CoV-2 for 48 hours, and then the cell viability were measured. Data are shown as mean ± SD and are representative of three (FIGS. 2A and 2B) independent experiments or the two replicate screens (FIG. 2C). *p < 0.05; **p < 0.01; ***p < 0.001. NS, not significant. ND, not detected. Analysis was performed using one-way ANOVA.

FIGS. 3A-J illustrate that Neu5Ac2en-OAcOMe exerts anti-viral effects in human cell models. FIG. 3A shows THP-1 cells were pretreated with vehicle or Neu5Ac2en-OAcOMe and were inoculated with HCoV-OC43 (MOI = 2) at either 4° C. (upper) or 37° C. (lower). Intracellular viral RNA was analyzed by RT-qPCR. FIGS. 3B-D show HCoV-OC43 (MOI = 2) infected THP-1 cells were treated with a gradient concentration of Neu5Ac2en-OAcOMe for 72 hours. Intracellular viral RNA (FIG. 3B, upper), extracellular progeny virus yields (FIG. 3B, lower and FIG. 3C) and intracellular nucleocapsid (FIG. 3D) were analyzed by RT-qPCR, TCID₅₀ assay and immunoblot, respectively. The inhibitory and cytotoxic curves (3C) were obtained using the data from the lower panel in FIG. 3B and cell viability measured by MTS assay. CellTiter 96® AQueous One Solution Cell Proliferation Assay (MTS) (Promega, G3582) was performed as instructed in the kit manual. FIG. 3E shows HCoV-OC43-infected THP-1 cells were treated with vehicle or Neu5Ac2en-OAcOMe for 24 hours. Immunoprecipitation was performed to capture nucleocapsid using anti HCoV-OC43-N antibody. Sialylation was detected with biotin-MAA and biotin-SNA lectins. FIG. 3F shows THP-1 cells infected with HCoV-OC43 were treated with or without Neu5Ac2en-OAcOMe and MG132. Nucleocapsid in the cell lysates was measured by western blot. FIG. 3G shows THP-1 cells infected with HCoV-OC43 and treated with or without Neu5Ac2en-OAcOMe were immunoprecipitated with an anti-N Ab and blotted for ubiquitin (FK2 Ab), and anti-N antibodies. FIGS. 3H and 3I show sialidase activity after incubation with Neu5Ac2en-OAcOMe (FIG. 3H) or oseltamivir (FIG. 31). FIG. 3J shows BSC-1 (MOI = 0.1) cells were inoculated with HCoV-OC43 virus for 2 hours, and Neu5Ac2en-OAcOMe was added after removal of inoculum. The levels of intracellular nucleocapsid were analyzed by immunofluorescence (48 hours post-infection). Red indicates nucleocapsid and blue represents cell nuclei stained by DAPI. Data are representative of at least three independent experiments. *p < 0.05; **p < 0.01; ***p < 0.001. NS, not significant. ND, not detected. Analysis was performed using one-way ANOVA (FIGS. 3A-3B) or unpaired Student’s t-test (FIGS. 3H-3I).

FIGS. 4A-G show the evaluation the therapeutic effects of Neu5Ac2en-OAcOMe. FIGS. 4A-E show the results of even-day-old mice were pre-treated with Neu5Ac2en-OAcOMe or vehicle and then IP infected with 30 µl virus dilution (1 x 10⁵ TCID₅₀ HCoV-OC43). FIG. 4A shows survival curves after HCoV-OC43 infection. (n=11 for vehicle group, n=10 for Neu5Ac2en-OAcOMe group). FIG. 4B shows body weight after HCoV-OC43 infection. (n=11 for vehicle group, n=10 for Neu5Ac2en-OAcOMe group). FIG. 4C shows viral RNA copies of HCoV-OC43 in the blood, brain and lung day 5 post-infection. (n=6 for vehicle group, n=7 for Neu5Ac2en-OAcOMe group). FIG. 4D shows cytokines in blood day 5 post-infection. (n=6 for vehicle group, n=7 for Neu5Ac2en-OAcOMe group). FIG. 4E is a histological analysis of brain and lung tissues day 5 post-infection. Tissue sections were stained with H&E. FIGS. 4F-G show the results of 5- to 6-week-old K18-hACE2 transgenic mice injected (IP) with vehicle control (n = 8) or Neu5Ac2en-OAcOMe and Neu5Ac2en9N3-OAcOMe (n=12) on days 0, 1, 2, 3 and 4 after infection with SARS-CoV-2 (1 × 10⁴ pfu/mouse). FIG. 4F shows survival of K18-hACE2 transgenic mice infected with SARS-CoV-2. Numbers above bars indicate the number of viable mice out of the total number of mice used per group. FIG. 4G shows body weight change after challenge with SARS-CoV-2. Data are representative of at least three independent experiments (FIGS. 4A-E). *p < 0.05; **p < 0.01; ***p < 0.001. Analysis was performed using Kaplan Meier analysis (FIG. 4A), unpaired Student’s t-test (FIGS. 4B-C), or one-way ANOVA (FIG. 4D).

### DETAILED DESCRIPTION

The present disclosure may be understood more readily by reference to the following detailed description of desired embodiments included therein. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All publications, patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It should be recognized that, as used herein, the term sialidase inhibitor also includes pharmaceutically acceptable salts thereof. The term “pharmaceutically acceptable salts” connotes salts commonly used to form alkali metal salts and to form addition salts of free acids or free bases. Suitable pharmaceutically acceptable acid addition salts may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, galactaric, and galacturonic acids.

Suitable pharmaceutically acceptable base addition salts include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used to form base addition salts. All of these salts may be prepared by conventional means from the corresponding sialidase inhibitor by reacting, for example, the appropriate acid or base with the sialidase inhibitor.

Prodrugs that are converted into sialidase inhibitors in vivo are also contemplated.

It should also be recognized that the antiviral compositions of the present disclosure may contain a pharmaceutical carrier and/or diluent(s). The compositions may also contain other agents such as, but not limited to, corn oil, dimethylsulfoxide, gelatin capsules, and other carriers.

The article titled “Targeted intracellular Neu1 for coronavirus infection treatment” by Yang et al., iScience 26, published online Jan. 23, 2023, DOI: https://doi.org/10.1016/j.isci.2023.106037 is incorporated by reference herein in its entirety and a copy is also included in an Appendix hereto.

There is an urgent need to better understand the virulence mechanisms of SARS-CoV-2 and the host response in order to develop therapeutics. The inventors of the present application used serum from human COVID-19 patients, cell lines infected with human coronavirus (HCoV-OC43) and a mouse model of HCoV-OC43 infection to demonstrate the critical role of sialylation in coronavirus replication. Significant sialylation of coronavirus nucleocapsid (N) protein was observed from both patients with COVID-19 and coronavirus HCoV-OC43-infected cells. Nucleic acid-binding assays and RT-qPCR revealed N protein sialylation controlled the RNA binding activity and replication of coronavirus, respectively. It was found that HCoV-OC43 infection significantly increased neuraminidase 1 (Neu1) expression, a regulator of sialylation. Neu1 overexpression in cells increased HCoV-OC43 replication, whereas Neu1 knockdown reduced HCoV-OC43 replication. Notably, Neu1 inhibitor Neu5Ac2en-OAcOMe selectively targets intracellular sialidase and significantly reduced HCoV-OC43 replication in vitro and rescued mice from HCoV-OC43 infection-induced death. These findings suggest that Neu1 inhibitors could be used to limit SARS-CoV-2 replication in patients with COVID-19, making Neu1 a potential therapeutic target for COVID-19 as well as future pandemics of coronavirus infection.

A new pathway of coronavirus replication, a new mechanism to regulate coronavirus replication, a new drug target to regulate coronavirus replication, and new therapeutic approach for coronavirus infection, especially for COVID-9 caused by SARS-CoV-2 virus are disclosed herein.

Glycosylation such as sialylation of coronavirus nucleocapsid (N) protein is a new mechanism for coronavirus replication.

Enzymes such as sialidases for glycosylation of coronavirus nucleocapsid (N) protein are new drug targets for inhibition of coronavirus replication.

Inhibitors of enzymes such as sialidases can be used as drugs for inhibition of coronavirus replication.

In some embodiments, the sialidase inhibitors are membrane-permeable and cytosolic sialidase inhibitors.

The sialidase inhibitors may be hydrophobic and lipophilic. They may have higher cell uptake and target cytosolic sialidases.

2-Deoxy-2,3-didehydro-N-acetylneuraminic acid (Neu5Ac2en or DANA) is an analogue of N-acetylneuraminic acid (Neu5Ac) and a pan sialidase(neuraminidase) inhibitor. The structural formula of this compound is provided below:

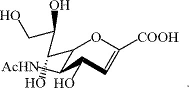

In some embodiments, the sialidase inhibitor is of the general formula (I)

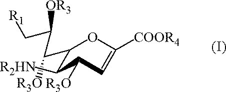

wherein R₁ is selected from the group consisting of OH, O-acetyl, N₃, OCOCH₂X₁ and NHCOCHX₁; X₁ is selected from the group consisting of H, a C₁-C₅ alkyl group, acetyl, propionyl, butyryl, valoryl, hexanoyl, pentanoyl, and 2-methylpropanyl; R₂ is H, acetyl, or COCH₂X₂; X₂ is selected from the group consisting of H, a C₁-C₅ alkyl group, acetyl, propionyl, butyryl, valoryl, hexanoyl, pentanoyl, and 2-methylpropanyl; R₃ is selected from the group consisting of H, OH, acetyl, COCH₂X₃ and OCOCH₂X₃; X₃ is selected from the group consisting of H, a C₁-C₅ alkyl group, acetyl, propionyl, butyryl, valoryl, hexanoyl, pentanoyl, and 2-methylpropanoal; R₄ is selected from the group consisting of H, OH, a C₁-C₆ alkyl group, and CH₂X₄; and X₄ is H or a C₁-C₆ alkyl group.

In particular embodiments, the sialidase inhibitor is of the following formula:

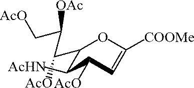

wherein Me is methyl and Ac is acetyl.

Those skilled in the art will be able to select an appropriate dosage. In non-limiting embodiments, the sialidase inhibitor(s) may be administered in an amount of from about 1 mg per kg body weight to about 50 mg per kg body weight, including from about 5 mg per kg to about 40 mg per kg, from about 10 mg per kg to about 30 mg per kg, from about 15 mg per kg to about 25 mg per kg, and about 20 mg per kg.

The following examples are provided to illustrate the devices and methods of the present disclosure. The examples are merely illustrative and are not intended to limit the disclosure to the materials, conditions, or process parameters set forth therein.

### EXAMPLES

### Methods

### Reagents

COVID-19 patient sera (both IgG and IgM antibodies to the N protein were negative) were purchased from Raybiotech (Peachtree, GA). Anti-SARS-CoV-2 N protein (HL5410, Catalog # MA5-36270) was obtained from Thermo Fisher Scientific (Waltham, MA), Anti-HCoV-OC43 N protein and anti-human Neu1 anti-antibodies were purchased from Sigma-Aldrich (St. Louis, MO). Anti-ubiquitin mouse monoclonal antibody (FK2) (cat. no. ST1200, lot no.D00165221) was obtained from EMD Millipore (Merck KGaA, Darmstadt, Germany). MG132 (cat. no. 3175-v, lot no. 640311) was purchased from Peptide Institute (Osaka, Japan). Biotinylated Maackia Amurensis Lectin II (MAL II, MAA) (cat. no. B-1265) and Biotinylated Sambucus Nigra Lectin (SNA, EBL) (cat. no. B-1305) were purchased from Vector Laboratories (Burlingame, CA). Anti-β-actin, Streptavidin-HRP and Horseradish peroxidase conjugated anti-mouse, anti-goat or anti-rabbit secondary antibodies were purchased from Santa Cruz Biotechnology (Santa Cruz, CA). Human Neu1 shRNAs were purchased from Sigma. HeLa, BSC-1, HEK293T and THP-1 cells were obtained from ATCC (Manassas, VA) and cultured in Dulbecco’s Modified Eagle Medium (DMEM) or Roswell Park Memorial Institute (RPMI) supplemented with 10% heat-inactivated fetal bovine serum, 2 mM glutamine, and 100 µg/ml penicillin/streptomycin. Neuraminidase (sialidase) provided by Vibrio cholerae (cat. no. 11080725001) were purchased from Sigma. The 32 m ssDNA (5′-CGAGGCCACGCGGAGTACGATCGAGGGTACAG-3′) was purchased from Thermo Fisher Scientific. The 32 m ssRNA 5′-CGAGGCCACGCGGAGUACGAUCGAGGGUACAG-3′) was purchased from Eurofins Genomics (Louisville, KY). SARS-CoV-2 nucleocapsid encoding plasmid was purchased from Sino Biological (cat. No. VG40588-UT, Beijing, China). Oseltamivir, zanamivir and Neu5Gc2en were obtained from Thermo Fisher Scientific. Neu5Ac2en9N3, Neu5Ac2en-OMe, Neu5Ac2en-OAcOMe and Neu5Ac2en9N3-OAcOMe were synthesized.

### Construction of Plasmids

To generate the constructs expressing human Neu1, cDNA for Neu1 was amplified by RT-PCR and subcloned into expression vectors pcDNA6 and pLVX-puro (Life Technologies, Carlsbad, CA). All constructs were verified by restriction enzyme digestion and DNA sequencing.

### Gel-mobility-shift Assay

ssDNA or ssRNA in phosphate buffer (10 mM sodium phosphate, 50 mM NaCl, 1 mM EDTA, 0.01% NaN3, pH 7.4) was heated to 95° C. and immediately put on ice to destroy its secondary structure. 1 x 10⁷ HEK293T cells in a 10 cm dish transfected with empty vector or SARS-CoV-2 N protein expression vector for 48 hours were harvested and suspended in lysis buffer (20 mM Tris-HCl, 0.1 % Triton X-100, 150 mM NaCl, pH 7.6) and then separated equally. Half of the cell lysates were treated with sialidase for 2 hours at 37° C. The oligonucleotides were mixed with the cell lysates and incubated on ice for 10 min and then separated on 1% agarose gels.

### Experimental Animal Models

WT C57BL/6J mice were obtained from Jackson Laboratory. All animal procedures were approved by the Animal Care and Use Committee of University of Tennessee Health Science Center. The HCoV-OC43 infection mouse model was established. Briefly, seven-day-old mice were separated randomly into two groups and injected intraperitoneally (IP) with either Neu5Ac2en-OAcOMe (20 mg/kg) or vehicle (0.5% dimethyl sulfoxide, DMSO). One hour later, mice were inoculated with 30 µl of virus dilution (1 x 10⁵ TClD₅₀ of HCoV-OC43) by IP injection. Neu5Ac2en-OAcOMe and vehicle were administered daily and mice were monitored up to 10 days for survival. To detect viral RNA loads in tissues and cytokine production, mice were euthanized at 5 days post-infection. Mouse brain, lung, and blood tissues were collected.

### Immunofluorescence

Cells were fixed with 4% paraformaldehyde in phosphate buffered saline (PBS) at room temperature for 15 min and then permeabilized with 1% Triton X-100 in PBS at room temperature for 15 min. Immunofluorescence staining was performed. Images were acquired with an EVOS FL Auto Imaging System (Thermo Fisher Scientific).

### HCoV-OC43 Production and Titration

HCoV-OC43 virus (ATCC® VR-1558™) was purchased from ATCC. The stock of HCoV-OC43 was produced and titrated using BSC-1 cells. Viral titers in cell-free culture supernatants were determined by endpoint dilution-based TClD₅₀ assays in 96-well plates. Cytopathic effect was recorded and used for calculation of viral titers at 7 days post-infection.

### SARS-CoV-2 Culture

The SARS-CoV-2 isolate USA-WA1/2020 was obtained through BEI Resources (NIAID, NIH: SARS-Related Coronavirus 2, Isolate USA-WA1/2020, NR-52281) and amplified in Vero-E6 cells (ATCC, VERO C1008) at an MOI of 0.1 in Minimal Essential Medium (MEM; Corning, 17-305-CV) supplemented with 5% heat-inactivated FBS (GIBCO) and 1% L-Glutamine (Corning, 25-005-CI) and 5 mM penicillin/streptomycin (GIBCO, 30-001-Cl). Following virus amplifications, viral titer was determined using a plaque assay using the method described previously for alphaviruses. All experiments involving SARS-CoV-2 were done in a biosafety level 3 laboratory and provided by a fee service by the University of Tennessee Health Science Center Regional Biocontainment BSL3 Laboratory.

### SARS-CoV-2 High-throughput Screen (HTS) Cytopathic Effect Assay

Double-blinded SARS-CoV-2 high-throughput screen (HTS) cytopathic effect assay were performed with the fee service provided by the University of Tennessee Health Science Center Regional Biocontainment BSL3 Laboratory. Briefly, the 7 sialidase inhibitors (oseltamivir, zanamivir, Neu5Gc2en, Neu5Ac2en9N3, Neu5Ac2en-OMe, Neu5Ac2en-OAcOMe and Neu5Ac2en9N3-OAcOMe) were plated in 384-well black wall plates containing 4,500 Vero 76 cells/well in single dose of indicated concentration in Eagle’s minimum essential medium with 5% heat inactivated FBS, 1% penicillin/streptomycin/L-glutamine, 1% Hepes and 0.5% DMSO. The cells were infected with SARS-CoV-2 at an MOI of 0.1. Plates were then allowed to incubate at 37° C., 5% CO2, for 48 h. The cell viability at the end of incubation period was measured. After incubation, 100 µL of Promega CellTiter-GloR (Promega, Madison, WI) was added to each well using the BiomekR 2000. Plates were shaken for 2 min at speed 5 on a Labline Instruments (Kochi, India) plate shaker. Luminescence was then measured using a PerkinElmer Envision™ plate reader (PerkinElmer, Wellesley, MA).

### SARS-CoV-2 Infection in Mice

Mice SARS-CoV-2 infection experiments were performed with the fee service provided by the University of Tennessee Health Science Center Regional Biocontainment BSL3 Laboratory. Age- and gender-matched, 5- to 6-week old K18-ACE-2 transgenic mice were anesthetized with 5% isoflurane and then infected intranasally with SARS-CoV-2 in 50 µL DPBS containing around 1 x 10⁴ PFU. Day 0, mice were administered intraperitoneally 200 µL of DPBS containing 100 µg of Neu5Ac2en-OAcOMe plus 100 µg of Neu5Ac2en9N3-OAcOMe two hours before infection. Infected mice continued to administer intraperitoneally 200 µL of DPBS containing 100 µg of Neu5Ac2en-OAcOMe plus 100 µg of Neu5Ac2en9N3-OAcOMe on days 1, 2, 3 and 4 post-infection. Mice were monitored over a period of 14 days for survival.

### Neuraminidase Activity Assay

Sialidase activity was measured using 2′-(4-methylumbelliferyl)-α-D-N-acetylneuraminic acid sodium salt hydrate (4-MU-NANA, catalog no. sc-222055, Santa Cruz Biotechnology) as the substrate. 1 x 10⁷ HEK293T cells in a 10 cm dish transfected with Neu1 expression vector were harvested after 48 hours, incubated with inhibitors for 30 min at room temperature, washed to remove the sialidase inhibitors, and separated equally. Half of the cells were used for detection of cell surface sialidase activity and half of the cells were suspended in lysis buffer (20 mM Tris-HCl, 0.1 % Triton X-100, 150 mM NaCl, pH 7.6) for detection of whole cell sialidase activity. For the reaction, intact cells or lysed cells were incubated with 4-MU-NANA (final concentration, 15 µM) for 30 min at 37° C. in 50 µl reaction buffer (50 mM Sodium phosphate, pH 5.0). The reaction was terminated by adding 600 µl stop buffer (0.25 M glycine-NaOH, pH 10.4). Fluorescence intensity was measured with a Synergy HTX Multi-Mode Reader (EMD Millipore, Merck KGaA) (excitation 360 nm; emission 460 nm).

### Real-time Quantitative PCR

Total RNA was extracted with TRIzol (Invitrogen, Carlsbad, CA) according to the manufacturer’s protocol and reverse transcribed with random primers and Superscript III (Life Technologies). The mRNA expression of human Neu1, Neu2, Neu3 and Neu4 was measured by real-time PCR. Samples were run in triplicate, and the relative expression was determined by normalizing the expression of each target to the endogenous reference, GAPDH. For RT-qPCR, copy numbers of HCoV-OC43 viral RNA were calculated based on a standard curve generated using pEF6-OC43-N-V5 His DNA. The following primers were used: Neu1: 5′-GGAGGCTGTAGGGTTTGGG-3′ (forward), 5′-CACCAGACCGAAGTCGTTCT-3′ (reverse); Neu2: 5′-CCATGCCTACAGAATCCCTGC-3′ (forward), 5′-CTCTGCGTGCTCATCCTTC-3′ (reverse); Neu3: 5′-AAGTGACAACATGCTCCTTCAA-3′ (forward), 5′-TCTCCTCGTAGAACGCTTCTC-3′ (reverse); Neu4: 5′-GGCCACGGGATGACAGTTG-3′ (forward), 5′-CAGGCGGATACCCATGTGAG-3′ (reverse); HCoV-OC43 N gene, 5′-CGATGAGGCTATTCCGACTAGGT-3′ (forward) and 5′-CCTTCCTGAGCCTTCAATATAGTAACC-3′ (reverse).

### Immunoblotting

Cell lysates were prepared in lysis buffer (20 mM Tris-HCl, 150 mM NaCl, 1 % Triton X-100, pH 7.6, including protease inhibitors, 1 µg/ml leupeptin, 1 µg/ml aprotinin and 1 mM phenylmethylsulfonyl fluoride), sonicated, centrifuged at 13,000 rpm for 5 min and then analyzed by Western blot. The concentration of running gel was 10%. After blocking, the blots were incubated with the appropriate primary antibody (1: 1,000 dilution) or Biotin-MAA/SNA (1 µg/ml). After incubation with the secondary antibody (HRP conjugated goat anti-rabbit IgG, goat anti-mouse IgG, 1: 5,000 dilution), or Streptavidin-HRP (1: 10,000 dilution), the signal was detected with an enhanced chemiluminescence (ECL) kit (Santa Cruz).

### Measurement of Inflammatory Cytokines

Mouse blood samples were obtained at indicated time points, and cytokines in the serum were determined using a mouse cytokine bead array designed for inflammatory cytokines (552364, BD Biosciences, San Jose, CA). Human cytokines in cell culture derived supernatants were determined using a human cytokine bead array designed for inflammatory cytokines (551811, BD Biosciences).

### Statistical Analysis

GraphPad Prism software (San Jose, CA) was used for data analysis. Data are shown as mean ± SD or mean ± SEM. Statistical significance was analyzed by two-tailed t-test for two groups or one-way analysis of variance (ANOVA) or two-way ANOVA for three or more groups. Differences in survival rates were analyzed by Kaplan-Meier plot and statistical significance was determined using a log-rank (Mantel-Cox) test. *P<0.05, **P<0.01, ***P<0.001, n.s., not significant.

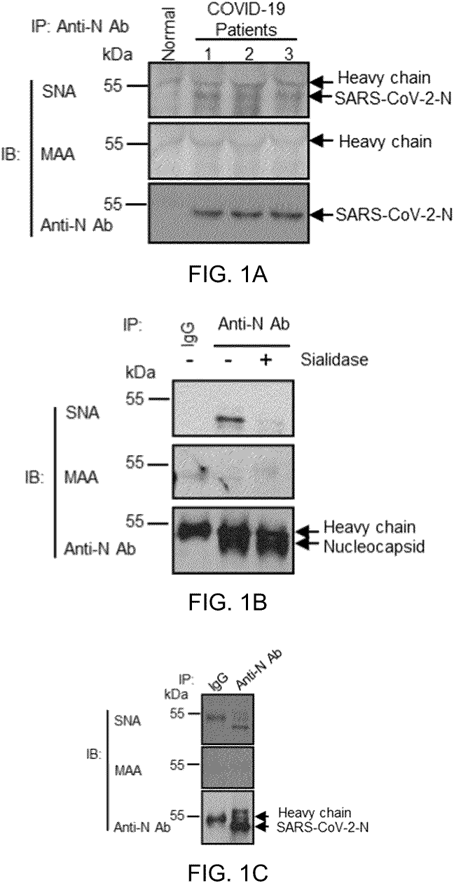

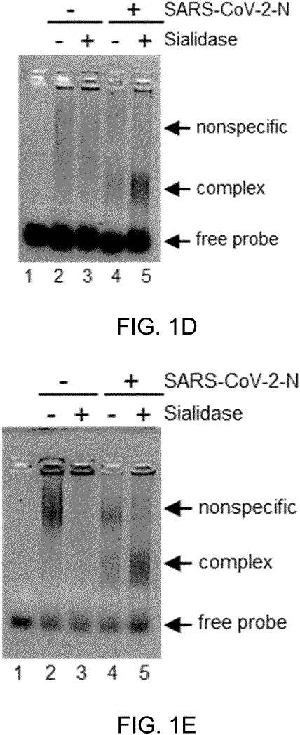

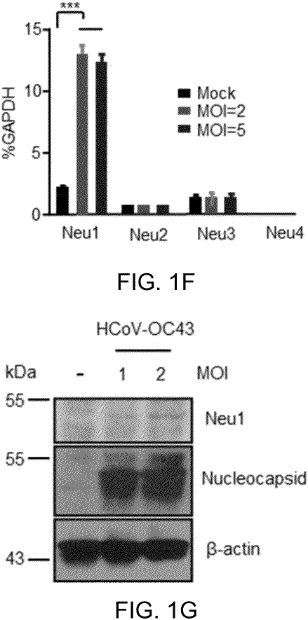

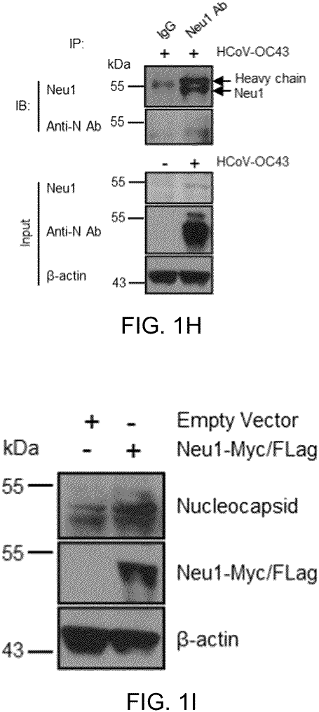

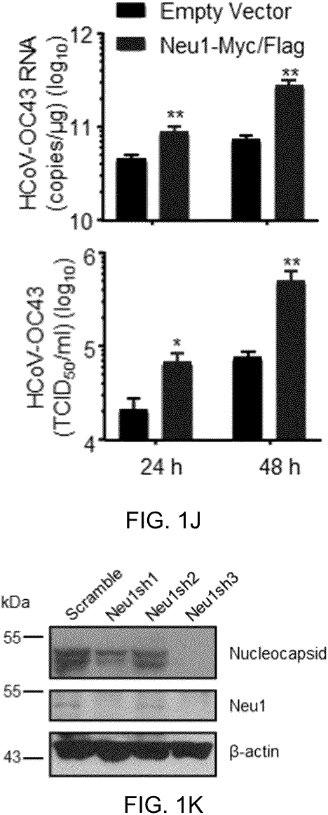

To determine whether sialylation occurred on N protein, lectin blot with immunoprecipitated samples from serum of COVID-19 patients and normal human was performed, and cell lysates infected with HCoV-OC43 using anti-N protein antibodies, which were treated with or without sialidase. As shown in FIGS. 1A and 1B, N protein from both patients with COVID-19 and HCoV-OC43-infected cells were heavily sialylated. The sialic acid was mostly attached in α 2,6 linkage on N protein (FIGS. 1A and 1B), and N protein sialylation was confirmed by sialidase treatment (FIG. 1B). In addition, sialylation was also observed on SARS-CoV-2-N protein expressed in HEK293T cells (FIG. 1C) and HCoV-OC43-N protein in THP-1 cells and HCoV-OC43 virion. Similar levels of sialylation were observed on cellular N protein and N protein in HCoV-OC43 virion, indicating that virus budding did not affect sialylation of N protein.

Since the primary role of N protein is to assemble with genomic RNA into the viral RNA-protein complex, whether the sialylation on N protein affects its RNA binding activity was investigated. To assess the nucleic acid-binding affinity of N protein, nucleic acid-binding assays were conducted in the presence of a 32-mer stem-loop II (32 m) motif single-stranded RNA (ssRNA) and its 32-mer ssDNA mimic. The 32 m ssRNA is a highly conserved sequence among coronaviruses and has been used to map the putative RNA-binding domain of SARS-CoV N protein. For nucleic acid-binding assays, HEK293T cells were lysed 48 hours after transfection with SARS-CoV-2-N protein expression vector and then treated the cell lysates with or without sialidase (Vibrio cholerae neuraminidase). SARS-CoV-2-N protein formed a strong complex with 32-mer ssRNA (FIG. 1D) and 32-mer ssDNA (FIG. 1E). As expected, HEK293T cell lysates transfected with empty vector did not form a complex with 32-mer ssRNA and ssDNA (FIG. 1D-1 E). Furthermore, 32-mer ssDNA and ssRNA bound to sialidase-treated N protein dramatically increased (FIGS. 1D-E). These findings indicate a significant increase in N protein RNA binding activity after sialidase treatment, supporting the critical role of N protein sialylation in RNA binding.

The sialylation level of a cell is largely dependent on the activity of two kinds of enzymes: sialyltransferases are responsible for adding sialic acid residues to glycolipids or glycoproteins, while sialidases are responsible for removing sialic acid residues from glycolipids or glycoproteins. The contribution of endogenous sialidases to the sialylation of N protein was evaluated using THP-1 cell lines. Real-time PCR (FIG. 1F) and western blot analysis (FIG. 1G) indicated the expression of Neu1 but not Neu2, Neu3 or Neu4 was significantly increased after infection with HCoV-OC43 for 72 hours. Notably, NEU1 also upregulated in COVID-19 patients. In addition, N protein associated with Neu1 in HCoV-OC43-infected cells (FIG. 1H). Sialylation level on N protein was significantly decreased in 293T cells over-expressing Neu1 compared with empty vector control cells.

The coronavirus N protein is a multifunctional RNA-binding protein necessary for viral replication. Since it was determined that the sialylation on N protein affects its RNA binding activity, it was investigated whether this sialylation affects virus replication. Viral infection was quantified by real-time quantitative PCR (RT-qPCR) with primers targeting the coding region of the viral N gene. RNA was collected from THP-1 cells at indicated time points after viral challenge and viral transcripts were quantified. Supernatants were also processed for quantification of viral titer by 50% tissue culture infective dose (TCID₅₀) assay. The replication of HCoV-OC43 was more than 10-fold higher at the level of viral transcripts and viral titers in cell culture supernatants (FIG. 1J) of cells overexpressing Neu1 (FIG. 11) than in cells expressing empty vector 48 hours after viral challenge. By contrast, the replication of HCoV-OC43 was more than 100-fold lower at the level of viral transcripts and viral titers in the cell culture supernatants (FIG. 1L) of cells overexpressing shRNA for Neu1 (FIG. 1K) than in cells expressing scrambled shRNA. Compared with scrambled shRNA, Neu1sh3 significantly decreased HCoV-OC43 replication more than Neu1sh1 and Neu1sh2, in consistence with the knockdown efficiency of shRNA (FIGS. 1K-L). N protein levels were also significantly decreased in Neu1 knockdown cells (FIG. 1K) but dramatically increased in Neu1 overexpressing cells (FIG. 1I). These data indicate host Neu1 is a regulator of HCoV-OC43 replication in THP-1 cells.

### Screening for Potential Sialidase Inhibitors

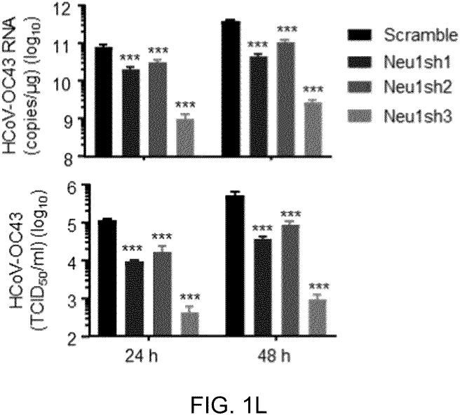

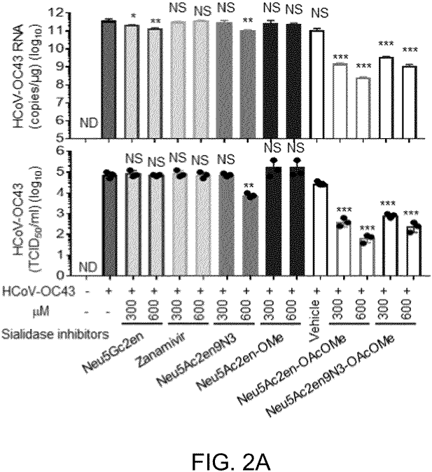

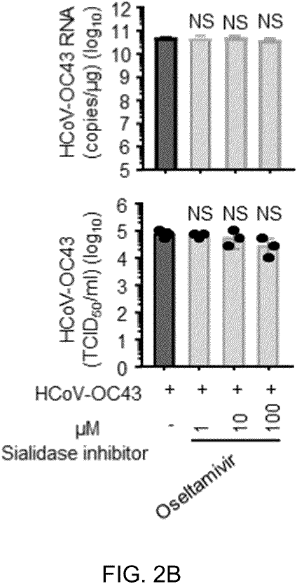

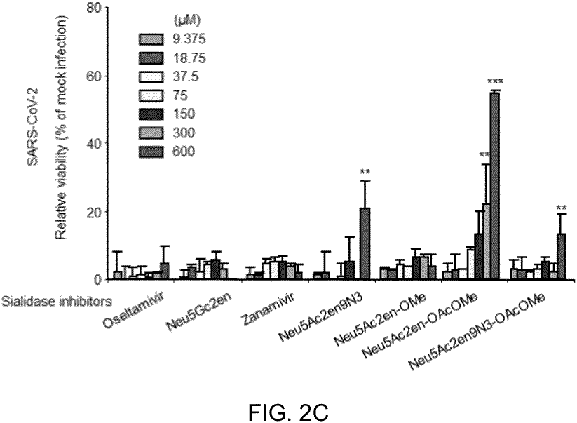

The significantly reduced HCoV-OC43 replication in Neu1 knockdown THP-1 cells suggests that endogenous sialidase plays a key role for HCoV-OC43 replication and thus may be a valuable therapeutic target. To test this hypothesis, commercially available sialidase inhibitors (oseltamivir, zanamivir and Neu5Gc2en) and synthetic sialidase inhibitors with different polarity (Neu5Ac2en9N3, Neu5Ac2en-OMe, Neu5Ac2en-OAcOMe and Neu5Ac2en9N3-OAcOMe) were assessed for antiviral activity against HCoV-OC43 in vitro. THP-1 cells treated with these inhibitors) were challenged with HCoV-OC43 for two hours, RNA was collected from cells and viral transcripts were quantified 72 hours after viral challenge (FIGS. 2A-B upper). Supernatants were also processed at 72 hours for quantification of viral titer by TCID₅₀ assay (FIGS. 2A-B lower). Three of the tested sialidase inhibitors significantly repressed viral replication (FIGS. 2A-B). Among them, hydrophobic Neu5Ac2en-OAcOMe showed the highest antiviral activities (FIG. 2A), which were dose-dependent. Next a high-throughput screen (HTS) assay was used to test whether these inhibitors have similar effect on the replication of SARS-CoV-2 and used cell viability 48 hours post SARS-CoV-2 infection as readout. Among them, Neu5Ac2en-OAcOMe also showed the highest protection activities (FIG. 2C), which is consistent with its highest antiviral activity against HCoV-OC43 above. In both assays, viral neuraminidase inhibitors zanamivir and oseltamivir did not show inhibitory activity (FIGS. 2A-C), which is in agreement with previous reports that zanamivir and oseltamivir have limited potency against all of the human sialidases.

### Antiviral Activity of Neu5Ac2en-OAcOMe

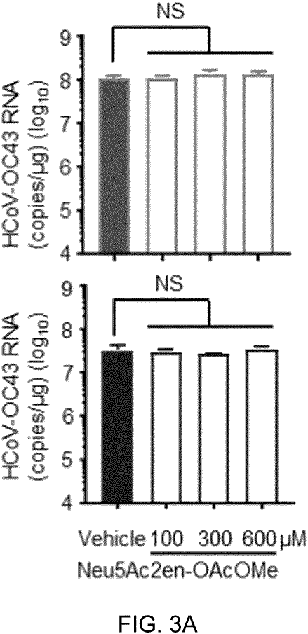

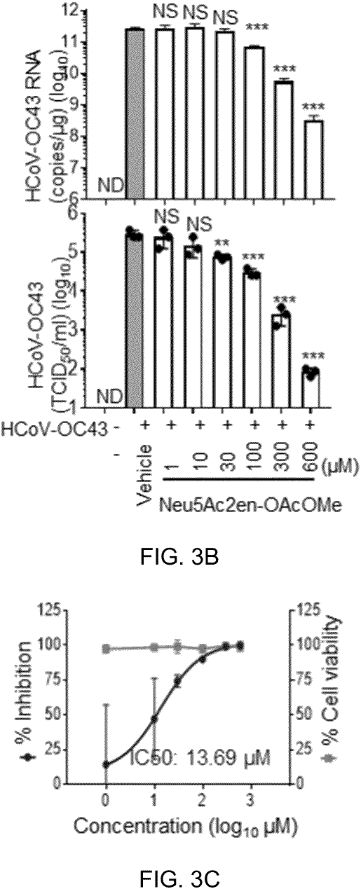

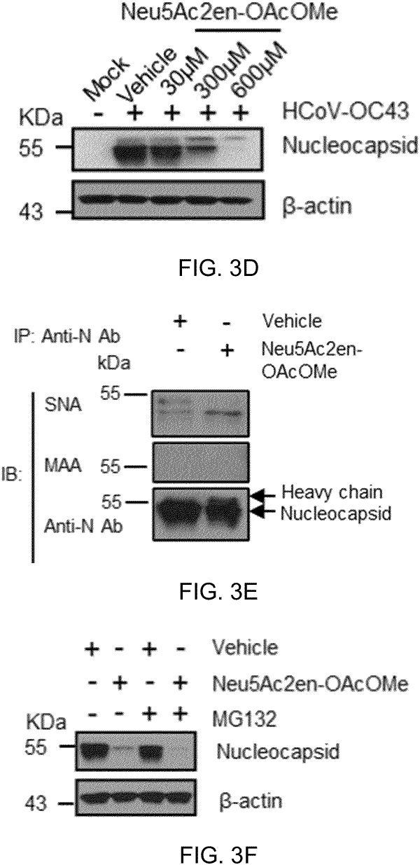

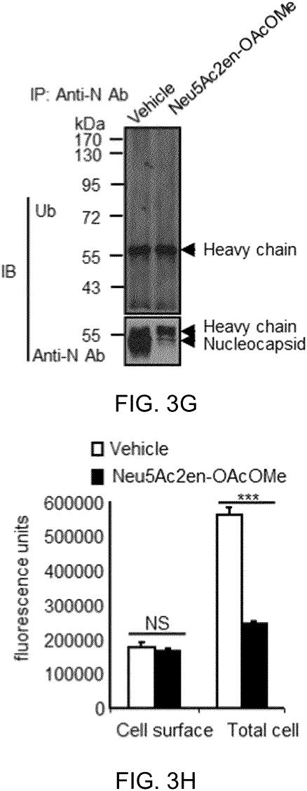

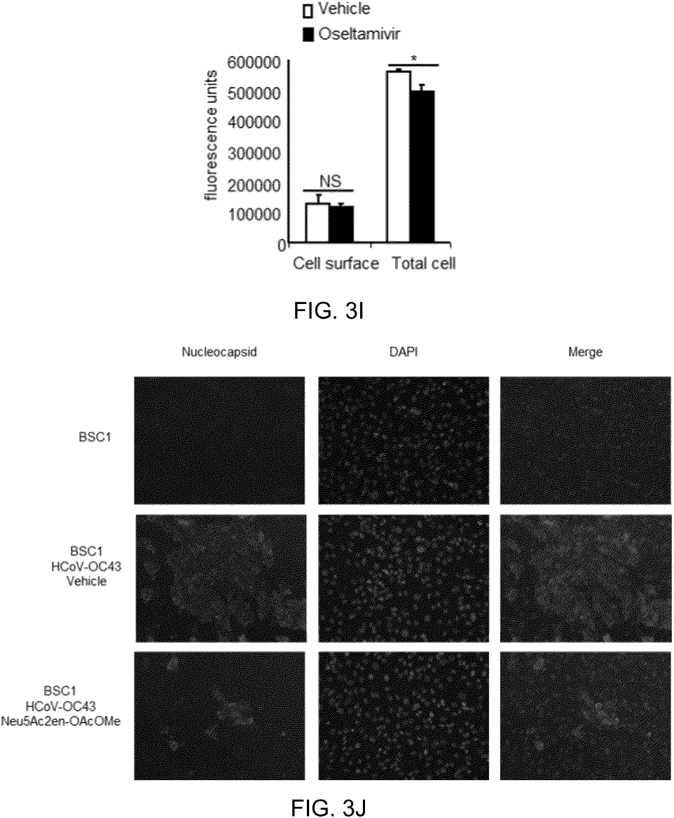

Whether Neu5Ac2en-OAcOMe acts on the virus binding and entry steps of the viral life cycle was evaluated. THP-1 cells were treated with Neu5Ac2en-OAcOMe for two hours and then infected the cells with HCoV-OC43 at 4° C. or 37° C. Cells incubated at 4° C. were collected one hour post-infection and cells incubated at 37° C. were collected two hours post-infection. Intracellular viral RNA was quantified by RT-qPCR. As shown in FIG. 3A, viral loads were similar among cells treated with different amounts of Neu5Ac2en-OAcOMe, indicating that Neu5Ac2en-OAcOMe treatment did not affect virus binding and entry steps of the viral life cycle.

To determine whether Neu5Ac2en-OAcOMe affects post-entry steps of the viral life cycle, THP-1 cells were infected with HCoV-OC43 for two hours and then treated the cells with Neu5Ac2en-OAcOMe. Intracellular viral RNA and viral titers were quantified in the cell culture supernatants 72 hours post-infection. Neu5Ac2en-OAcOMe treatment significantly decreased viral replication in THP-1 cells at the level of viral transcripts (FIG. 3B, upper) and viral titers in cell culture supernatants (FIG. 3B, lower) in a dose-dependent manner, with an IC₅₀ of 13.69 µM (FIG. 3C). N protein levels were also significantly decreased in Neu5Ac2en-OAcOMe-treated cells (FIG. 3D). Compared with vehicle-treated cells, Neu5Ac2en-OAcOMe-treated cells showed increased sialylation of HCoV-OC43 N protein (FIG. 3E). Importantly, Neu5Ac2en-OAcOMe was non-toxic at all tested concentrations in MTS assay (FIG. 3C) and did not induce cytokine production in THP-1 cells. Moreover, CID-1067700, a competitive inhibitor of Rab7 activation and can block β-coronavirus egress, moderately inhibited HCoV-OC43 release, but not viral RNA replication, at 24h post infection. CID-1067700 did not reverse the decrease of intracellular viral RNA and extracellular viral production triggered by Neu5Ac2en-OAcOMe treatment, suggesting the step of viral RNA replication is the target of Neu5Ac2en-OAcOMe.

To rule out the possibility that Neu5Ac2en-OAcOMe treatment induced protein degradation, the cells were treated with MG132. As shown in FIG. 3F, inhibiting proteasome activity by MG132 did not affect the decrease in N protein expression induced by Neu5Ac2en-OAcOMe. Neu5Ac2en-OAcOMe also did not alter the levels of ubiquitination on HCoV-OC43-N protein (FIG. 3G). Thus, decreased N protein expression was not likely due to protein degradation.

Very interestingly, Neu1 overexpression reversed the inhibition of Neu5Ac2en-OAcOMe on HCoV-OC43 infection, indicating Neu1 is the target of Neu5Ac2en-OAcOMe. Since Neu1 exists on the plasma membrane and cytoplasm, it was investigated whether Neu5Ac2en-OAcOMe-sensitive Neu1 resides on the plasma membrane or cytoplasm. To test this, HEK293T cells transfected with Neu1 expression vector and Neu5Ac2en-OAcOMe were incubated at room temperature for 30 min. Cell surface sialidase activity and total cell lysate sialidase activity were measured. Neu5Ac2en-OAcOMe significantly decreased total sialidase activity but did not affect cell surface sialidase activity (FIG. 3H), indicating that Neu5Ac2en-OAcOMe-sensitive Neu1 was located in the cytoplasm, where coronavirus replication takes place. By contrast, Neu5Gc2en targets sialidase on cell surface, which may explain why Neu5Gc2en did not inhibit HCoV-OC43 replication in THP-1 cells (FIG. 2A) and could not protect SARS-CoV-2 infection induced cell death (FIG. 2C). Consistent with previous research, oseltamivir treatment only slightly decreased sialidase activity in the cytoplasm (FIG. 3I).

Next, it was tested whether the observed efficacy of Neu5Ac2en-OAcOMe was not restricted to THP-1 cells. The efficacy of Neu5Ac2en-OAcOMe was evaluated in three epithelial cell lines (HEK293T cells, HeLa cells and BSC-1 cells). Neu5Ac2en-OAcOMe inhibited viral replication in all three cell lines and significantly decreased N protein levels. Immunofluorescence staining for HCoV-OC43-N protein also showed Neu5Ac2en-OAcOMe treatment effectively suppressed viral replication in BSC-1 cells (FIG. 3J). In summary, these results indicate the tested sialidase inhibitors did not inhibit the entry step of viral replication but interfered in the subsequent steps of the viral life cycle of coronavirus. Therefore, cytosolic sialidase inhibitors represent a potential treatment for coronavirus infections.

### Therapeutic Activity of Neu5Ac2en-OAcOMe

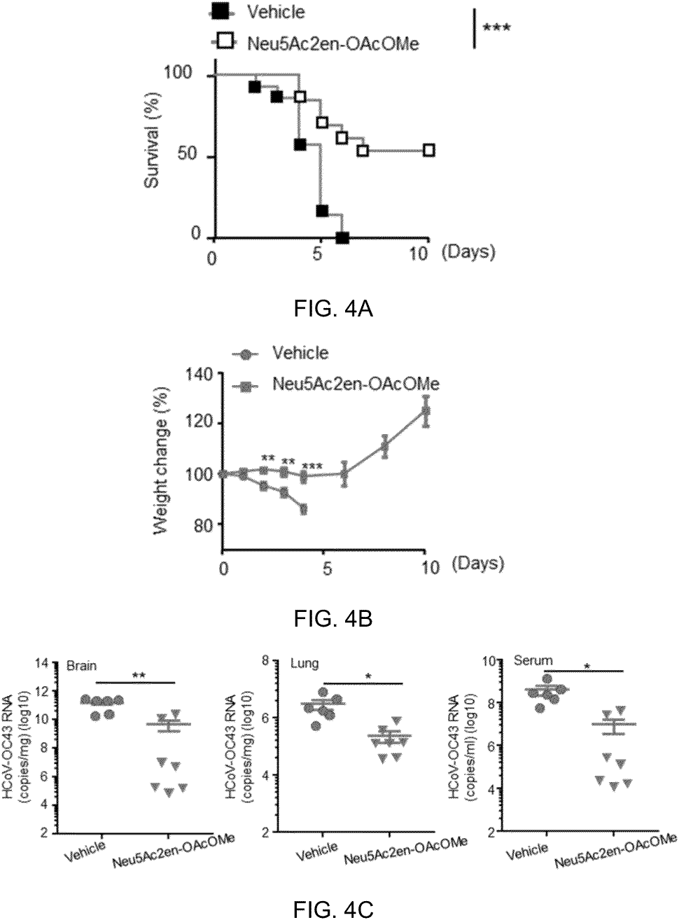

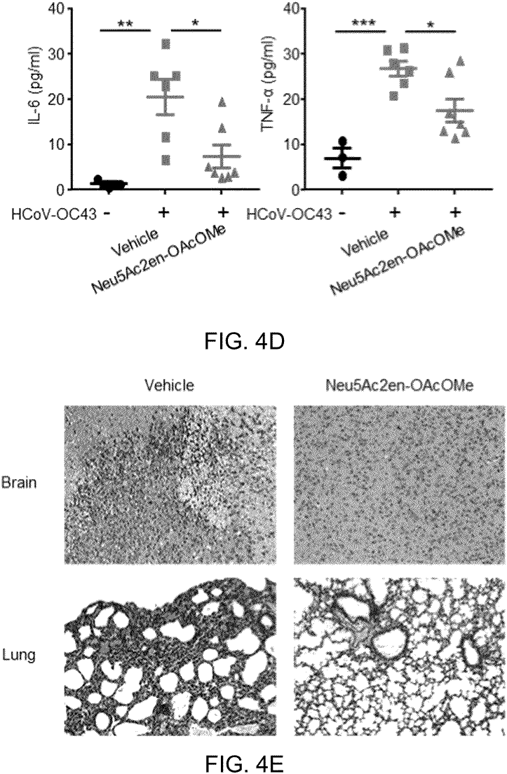

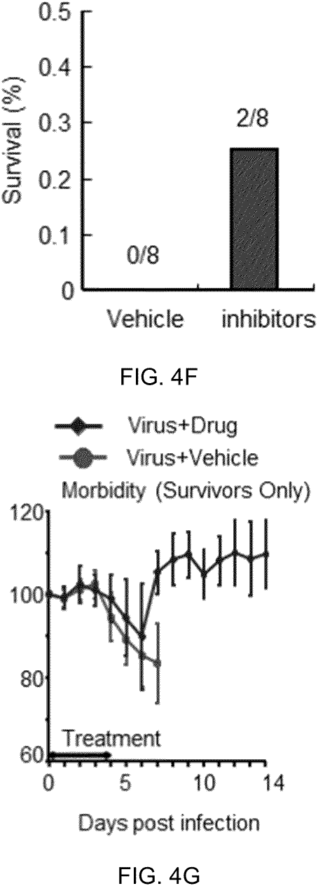

The antiviral efficacy of Neu5Ac2en-OAcOMe was examined in vivo. No toxic signs and symptoms were observed during all treatment period. It was examined whether Neu5Ac2en-OAcOMe could prevent HCoV-OC43 infection-induced death in newborn mice. Seven-day-old C57BL/6 pups were injected (intraperitoneal, IP) with 30 µl virus dilution (1 × 10⁵ TCID₅₀ of HCoV-OC43). Based on in vitro data, Neu5Ac2en-OAcOMe (20 mg/kg) was injected (IP) one hour before virus infection. As shown in FIG. 4A, 100% of vehicle-treated mice succumbed to HCoV-OC43 challenge, while 50% of Neu5Ac2en-OAcOMe-treated mice survived throughout the 10 days observation period. Body weight was significantly lower in vehicle-treated mice than in Neu5Ac2en-OAcOMe-treated mice (FIG. 4B). Neu5Ac2en-OAcOMe-treated mice also showed suppression of HCoV-OC43 viral replication in the lungs, blood and brain (FIG. 4C).

Both viral and host factors impact disease pathogenesis. During infection with SARS-CoV, Middle East respiratory syndrome coronavirus and SARS-CoV-2, cytokine storm is a major cause of mortality. In our previous study, sialidase inhibitors rescued mice from bacterial infection-induced death by inhibiting the activity of host Neu1 and suppressing the cytokine storm. To determine whether Neu5Ac2en-OAcOMe exerts similar effects in coronavirus infection, serum levels of interleukin 6 (IL-6) and tumor necrosis factor alpha (TNF-α) were measured, which contribute to the cytokine storm and correlate with respiratory failure and adverse clinical outcome in COVID-19. A substantial decrease of serum IL-6 and TNF-α levels was detected in Neu5Ac2en-OAcOMe-treated mice (FIG. 4D). Hematoxylin and eosin (H&E) staining also indicated that brain and lung tissue damage were improved in Neu5Ac2en-OAcOMe-treated mice (FIG. 4E).

To test the potential efficacy of this treatment during SARS-CoV-2 infection, a murine SARS-CoV-2 infection model was used. By 7 days post-infection, all vehicle control-treated mice succumbed to the infection. Conversely, treatment with Neu5Ac2en-OAcOMe provided significant protection against SARS-CoV-2-induced mortality (FIG. 4F) and body weight loss (FIG. 4G). Taken together, these findings show Neu5Ac2en-OAcOMe conferred protection against HCoV-OC43 and SARS-CoV-2 challenge by reducing viral replication in vivo and the associated inflammatory dysregulation.

### Dosage

Neu5Ac2en-OAcOMe (20 mg/kg) for HCoV-OC43 infection was administered in mice experiments.

100 µg of Neu5Ac2en-OAcOMe plus 100 µg of Neu5Ac2en9N3-OAcOMe per mouse for SARS-CoV-2 infection was administered in mice experiments.

### Discussion

N protein has a mass of 50 to 60 kDa (FIGS. 1 and 3), indicating the presence of post-translational modifications such as N, or O-linked glycosylation sialylation (FIG. 1). Moreover, SARS-CoV-2-N protein is highly glycosylated demonstrated by glycomic and glycoproteomic analyses after expression in HEK293T cells. N protein from SARS-CoV-2 and HCoV-OC43 was significantly sialylated and this sialylation was tightly regulated by host Neu1. Coronavirus replication occurs in cytoplasm of infected host cells. The sialidase inhibitor Neu5Ac2en-OAcOMe targets cytoplasmic sialidase but not cell surface sialidase, especially recent studies indicated that β-Coronaviruses traffic to lysosomes where Neu1 is also known to be predominantly localized. Notably, the newly developed cytosolic sialidase inhibitor Neu5Ac2en-OAcOMe reduced HCoV-OC43 and SARS-CoV-2 replication in vitro and in vivo by inhibiting host Neu1 activity.

Inhibition of viral neuraminidase activity has developed as a therapeutic approach for influenza infection. Tamiflu (oseltamivir) and Relenza (zanamivir), which are approved for treatment of influenza A and B, have almost no effect on human sialidases. Several clinical trials have assessed the efficacy of oseltamivir in treating SARS-CoV-2 infection, but no positive outcomes were observed. This lack of efficacy could be attributed to several reasons: 1) SARS-CoV-2 genomic RNA does not code sialidase; 2) SARS-CoV-2 replication depends on the host Neu1 (FIG. 1); and 3) oseltamivir has less inhibitory effects on host Neu1 (FIG. 2J). Together, these findings explain why oseltamivir has not shown efficacy in the treatment of COVID-19.

Overall, it was demonstrated that hydrophobic Neu5Ac2en-OAcMe is a membrane-permeable inhibitor of the Neu1 sialidase, which is readily converted to the corresponding Neu5Ac2en intracellularly upon entering into the cells. Neu5Ac2en then acts as effective sialidase inhibitor to shut down the desialylation of N protein inside of the cells infected with coronavirus. It was demonstrated that Neu5Ac2en-OAcOMe targeted cytosolic Neu1 sialidase in the host cells, accounting for the contributions of cytosolic desialylation to viral replication in the disease process. Based on these findings, cytosolic sialidase inhibitors could be a generalizable and effective treatment in the current COVID-19 pandemic as well as future coronavirus pandemics.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A method for treating a viral infection comprising:
administering an effective dose of an antiviral composition comprising a sialidase inhibitor.

2. The method of claim 1, wherein the sialidase inhibitor is of general formula (I): 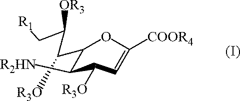
wherein R
₁ is selected from the group consisting of OH, O-acetyl, N₃, OCOCH₂X₁ and NHCOCHX₁; X₁ is selected from the group consisting of H, a C₁-C₅ alkyl group, acetyl, propionyl, butyryl, valoryl, hexanoyl, pentanoyl, and 2-methylpropanyl; R₂ is H, acetyl, or COCH₂X₂; X₂ is selected from the group consisting of H, a C₁-C₅ alkyl group, acetyl, propionyl, butyryl, valoryl, hexanoyl, pentanoyl, and 2-methylpropanyl; R₃ is selected from the group consisting of H, OH, acetyl, COCH₂X₃ and OCOCH₂X₃; X₃ is selected from the group consisting of H, a C₁-C₅ alkyl group, acetyl, propionyl, butyryl, valoryl, hexanoyl, pentanoyl, and 2-methylpropanoal; R₄ is selected from the group consisting of H, OH, a C₁-C₆ alkyl group, and CH₂X₄; and X₄ is H or a C₁-C₆ alkyl group.

3. The method of claim 1, wherein sialidase inhibitor is of the formula: 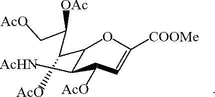
.

4. The method of claim 1, wherein the antiviral composition is administered orally.

5. The method of claim 1, wherein the antiviral composition is administered via an injection.

6. The method of claim 1, wherein the antiviral composition is administered intraperitoneally.

7. The method of claim 1, wherein the antiviral composition is administered intravenously.

8. The method of claim 1, wherein the viral infection is a coronavirus infection.

9. The method of claim 1, wherein the coronavirus infection is a severe acute respiratory syndrome coronavirus 2 (SARS-CoV-2) infection.

10. The method of claim 1, wherein the sialidase inhibitor is a membrane-permeable and cytosolic sialidase inhibitor.

11. The method of claim 1, wherein the sialidase inhibitor is hydrophobic and lipophilic.

12. A sialidase inhibitor of general formula (I): 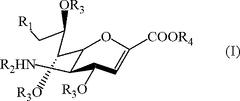
wherein R
₁ is selected from the group consisting of OH, O-acetyl, N₃, OCOCH₂X₁ and NHCOCHX₁; X₁ is selected from the group consisting of H, a C₁-C₅ alkyl group, acetyl, propionyl, butyryl, valoryl, hexanoyl, pentanoyl, and 2-methylpropanyl; R₂ is H, acetyl, or COCH₂X₂; X₂ is selected from the group consisting of H, a C₁-C₅ alkyl group, acetyl, propionyl, butyryl, valoryl, hexanoyl, pentanoyl, and 2-methylpropanyl; R₃ is selected from the group consisting of H, OH, acetyl, COCH₂X₃ and OCOCH₂X₃; X₃ is selected from the group consisting of H, a C₁-C₅ alkyl group, acetyl, propionyl, butyryl, valoryl, hexanoyl, pentanoyl, and 2-methylpropanoal; R₄ is selected from the group consisting of H, OH, a C₁-C₆ alkyl group, and CH₂X₄; and X₄ is H or a C₁-C₆ alkyl group.

13. The sialidase inhibitor of claim 12, wherein the sialidase inhibitor is of the formula 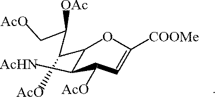
.

14. An antiviral composition comprising:
a carrier; and
a sialidase inhibitor.

15. The antiviral composition of claim 14, wherein the sialidase inhibitor is a membrane-permeable sialidase inhibitor.

16. The antiviral composition of claim 14, wherein the sialidase inhibitor is a cytosolic sialidase inhibitor.

17. The antiviral composition of claim 14, wherein the sialidase inhibitor is hydrophobic.

18. The antiviral composition of claim 14, wherein the sialidase inhibitor is lipophilic.

19. The antiviral composition of claim 14, wherein the sialidase inhibitor is of general formula (I): 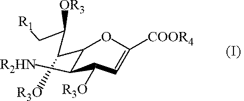
wherein R
₁ is selected from the group consisting of OH, O-acetyl, N₃, OCOCH₂X₁ and NHCOCHX₁; X₁ is selected from the group consisting of H, a C₁-C₅ alkyl group, acetyl, propionyl, butyryl, valoryl, hexanoyl, pentanoyl, and 2-methylpropanyl; R₂ is H, acetyl, or COCH₂X₂; X₂ is selected from the group consisting of H, a C₁-C₅ alkyl group, acetyl, propionyl, butyryl, valoryl, hexanoyl, pentanoyl, and 2-methylpropanyl; R₃ is selected from the group consisting of H, OH, acetyl, COCH₂X₃ and OCOCH₂X₃; X₃ is selected from the group consisting of H, a C₁-C₅ alkyl group, acetyl, propionyl, butyryl, valoryl, hexanoyl, pentanoyl, and 2-methylpropanoal; R₄ is selected from the group consisting of H, OH, a C₁-C₆ alkyl group, and CH₂X₄; and X₄ is H or a C₁-C₆ alkyl group.

20. The sialidase inhibitor of claim 14, wherein sialidase inhibitor is of the formula: 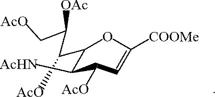
.
